(19) United States
(12) Patent Application Publication
(10) Pub. No.: US 2023/0413749 A1
(43) Pub. Date: Dec. 28, 2023

(54) Method for Inducing Lateral Branch Development of Eucheuma Denticulatum

(71) Applicants: Yazhou Bay Innovation Institute, Hainan Tropical Ocean University, Sanya (CN); Sanya Oceanographic Institute, Ocean University of China, Sanya (CN)

(72) Inventors: Yunxiang Mao, Sanya (CN); Zhendong Wang, Sanya (CN); Lei Tang, Sanya (CN); Yajian Zhang, Sanya (CN)

(21) Appl. No.: 18/215,475

(22) Filed: Jun. 28, 2023

(30) Foreign Application Priority Data
Jun. 28, 2022 (CN) 202210737558.2

(51) Int. Cl. A01G 33/00

(52) U.S. Cl. CPC A01G 33/00

(57) ABSTRACT

The present disclosure provides a method for inducing a lateral branch development of Eucheuma denticulatum, and relates to the technical field of marine organisms. In the present invention, the E. denticulatum is subjected to aeration culture at a light-photon flux of 200 μmol·m⁻²·s⁻¹ to 300 μmol·m⁻²·s⁻¹. During induction with a high light intensity, new lateral branches continue to grow; after the high-light treatment is completed, the E. denticulatum is placed in its original breeding environment. The dedifferentiation of branch cells into meristems is induced by the high light intensity in E. denticulatum, and then the meristems are developed into lateral branches. An increase of the lateral branches can increase a yield of E. denticulatum, and the high-light treatment can significantly increase a dry matter content of the E. denticulatum to raise an income.

### CROSS REFERENCE TO RELATED APPLICATION

This patent application claims the benefit and priority of Chinese Patent Application No. 202210737558.2, filed with the China National Intellectual Property Administration on Jun. 28, 2022, the disclosure of which is incorporated by reference herein in its entirety as part of the present application.

### TECHNICAL FIELD

The present disclosure belongs to the technical field of marine organisms, and in particular relates to a method for inducing a lateral branch development of Eucheuma denticulatum.

### BACKGROUND

Eucheumatoid seaweeds, including the genus of Kappaphycus, Eucheuma, and Betaphycus are the most productive species of macroalgae worldwide and used as raw material algae in industrial extraction of carrageenan. The Eucheuma is mainly cultivated in tropical-subtropical sea area such as Southeast Asia. China has the largest production of carrageenan in the world. However, the yield of Eucheuma in China has been shrinking annually and is remained at a low level for a long time. In 2020, the yield of Eucheuma in China is 3,856 tons (dry weight), accounting for only 0.147% of a total domestic production of macroalgae cultivation. The current situation of low yield lead to the raw material algae in China is almost entirely dependent on imports.

Eucheuma denticulatum is an important cultivated species in the Eucheuma genus. The morphology of E. denticulatum shows a structure of upright main body with multi-branches. At present, vegetative propagation is the main method in cultivation and breeding of E. denticulatum. However, long term vegetative reproduction reduces the genetic diversity and environmental adaptability of the population, resulting in the yield decreasing. Therefore, there is an urgent need for new farming models to improve the current situation.

Branching is an important trait in agricultural production. For example, tillering of gramineous crops is directly related to crop biomass and yield. Similarly, for multicellular red algae with multiple lateral branches, inducing the formation of lateral branches can significantly increase a yield of the E. denticulatum per unit of seawater. However, there is currently no method for the formation of lateral branches in multicellular red algae.

### SUMMARY

In view of this, an objective of the present disclosure is to provide a method for inducing a lateral branch development of Eucheuma denticulatum. The method can efficiently induce the formation of lateral branches in macroalgae of Eucheuma, thus improving the yield in per unit of seawater.

To achieve the above objective, the present disclosure provides the following technical solutions:

The present disclosure provides a method for inducing a lateral branch development of E. denticulatum, including the following steps: conducting aeration culture on the E. denticulatum under light illumination of 200 μmol·m⁻²·s⁻¹ to 300 μmol·m⁻²·s⁻¹.

Preferably, the aeration culture is conducted on a Provasoli's enrichment solution (PES) medium.

Preferably, the light illumination includes 12 h continuous light and 12 h continuous darkness every day.

Preferably, the aeration culture is conducted for at least 10 d.

Beneficial effects: the present disclosure provides a method for inducing a lateral branch development of E. denticulatum, including the following steps: conducting aeration culture on the E. denticulatum at a light-photon flux of 200 μmol·m⁻²·s⁻¹ to 300 μmol·m⁻²·s⁻¹. During induction with a high light intensity, new lateral branches continue to formation; after the high-light treatment completed, the E. denticulatum is cultivated in original environment. The dedifferentiation of branch cells into meristems is induced by the high light intensity in E. denticulatum, and then the meristems are formed into lateral branches. An increase of the lateral branches can increase a yield of E. denticulatum, and the high-light treatment can significantly increase a dry matter content of the E. denticulatum to raise an income.

### BRIEF DESCRIPTION OF THE DRAWINGS

FIG. 1 shows the number of cumulative developments of lateral branches induced under different light intensities; where a, b, and c represent significant differences in a cumulative increase of lateral branches per centimeter under different light intensities on the 10^{th} day;

FIG. 2 shows a dry matter content (%) on the 10^{th} day; where a, b, and c represent significant differences in the dry matter content under different light intensities on the 10^{th} day;

FIGS. 3A-B show states of a same E. denticulatum plant cultivated under different light intensities for 10 d; where FIG. 3A is under a light intensity of 50 μmol·m⁻²·s⁻¹, FIG. 3B is under a light intensity of 300 μmol·m⁻²·s⁻¹;

FIGS. 4A-B show states of a same E. denticulatum plant cultivated under different light intensities for 10 d; where FIG. 4A is under a light intensity of 50 μmol·m⁻²·s⁻¹, FIG. 4B is at a light-photon flux of 300 μmol·m⁻²·s⁻¹; and

FIGS. 5A-B show photos of E. denticulatum with different biomass levels after being treated at a light-photon flux of 300 μmol·m⁻²·s⁻¹ for 15 d.

### DETAILED DESCRIPTION OF THE EMBODIMENTS

The present disclosure provides a method for inducing a lateral branch development of E. denticulatum, including the following steps: conducting aeration culture on the E. denticulatum at a light-photon flux of 200 μmol·m⁻²·s⁻¹ to 300 μmol·m⁻²·s⁻¹.

In the present disclosure, lateral branches of E. denticulatum are induced under a high light intensity, and the induction is preferably conducted on a PES medium for at least 10 d. The PES medium includes preferably NaNO₃ 2.35 g/L, Na₂glycerophosphate·5H₂O 0.35 g/L, ES Fe solution 162.5 mL/L, P-II metal solution 162.5 mL/L, and HEPES buffer 3.25 g/L. There is no special limitation on a preparation method of the PES medium, and the preparation method includes preferably: adding the above raw materials into water and stirring in sequence, adjusting a pH value to 7.8, diluting to 1 L, sterilizing at 0.1 MPa and 121° C. for 20 min, and storing at 4° C. The ES Fe solution includes preferably components with the following concentrations: Fe(NH₄)₂(SO₄)₂·6H₂O 0.7 g/L and Na₂EDTA·2H₂O 0.6 g/L. The P-II metal solution includes preferably components with the following concentrations: Na₂EDTA·2H₂O 1 g/L, H₃BO₃ 1.14 g/L, FeCl₃·6H₂O 0.049 g/L, MnSO₄·H₂O 0.164 g/L, ZnSO₄·7H₂O 0.022 g/L, and CoCl₂·6H₂O 0.0048 g/L.

In the present disclosure, the induction includes preferably 12 h continuous light and 12 h continuous darkness every day while maintaining aeration culture. There is no special limitation on an aeration volume of the aeration culture. During the induction with a high light intensity, new lateral branches continue to grow; after the high-light treatment is completed, the E. denticulatum is placed in its original breeding environment. The dedifferentiation of branch cells into meristems is induced by the high light intensity in E. denticulatum, and then the meristems are developed into lateral branches. An increase of the lateral branches can increase a yield of E. denticulatum, and the high-light treatment can significantly increase a dry matter content of the E. denticulatum to raise an income.

The method for inducing a lateral branch development of E. denticulatum provided by the present disclosure is described in detail below with reference to the examples, but these examples may not be understood as a limitation to the protection scope of the present disclosure.

### Example 1

E. denticulatum was subjected to aeration culture at a light-photon flux of 50, 100, 200, and 300 μmol·m⁻²·s⁻¹ separately for 10 d, and four biological replicates were performed under each condition.

Determination of the initial number of lateral branches was recorded as a 0^{th} day, and the number of lateral branches per centimeter was counted every two days.

A calculation method for the number of induced lateral branches: the number of lateral branches on an n^{th} day/the length of the n^{th} day—the number of lateral branches on the 0^{th} day/the length of the 0^{th} day.

Determination of dry matter content: the weight of the algae after drying with absorbent paper was a fresh weight m₁, and the algae was dried in an oven at 60° C. until the weight constant, which was a dry weight m₂, and the dry matter content was: (m₂/m₁)×100%.

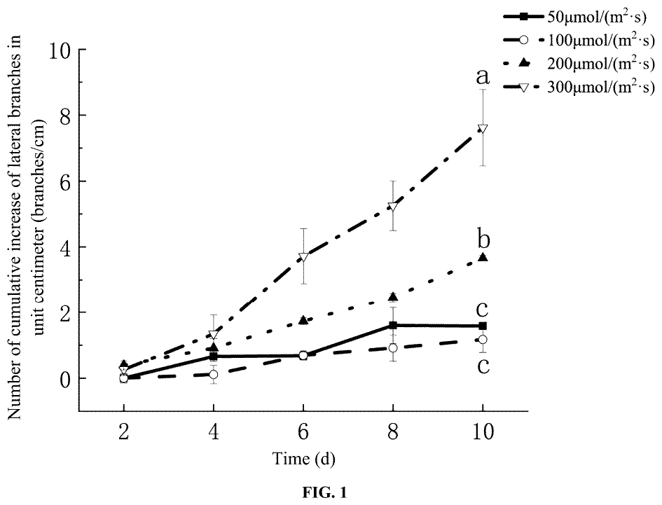

The number of lateral branches induced under different light intensities was shown in Table 1 and FIG. 1: on the 10^{th} day, the number of lateral branches per centimeter of algae at a light-photon flux of 300 μmol·m⁻²·s⁻¹ reached 7.62±1.16, which was significantly higher than the number of lateral branches increased under other light intensities (P<0.01). It showed that the high light-photon flux of 300 μmol·m⁻²·s⁻¹ could efficiently induce lateral branches in E. denticulatum.

TABLE 1
Cumulative number of lateral branches induced by different light intensities

| Light intensity | Day 2 | Day 4 | Day 6 | Day 8 | Day 10 |
|---|---|---|---|---|---|
| (μmol · m⁻² · s⁻¹) | (branches/cm) | (branches/cm) | (branches/cm) | (branches/cm) | (branches/cm) |
| 50 | 0.00 ± 0.00 | 0.66 ± 0.15 | 0.68 ± 0.11 | 1.60 ± 0.55 | 1.58 ± 0.08^{c} |
| 100 | 0.00 ± 0.14 | 0.11 ± 0.28 | 0.69 ± 0.14 | 0.91 ± 0.40 | 1.17 ± 0.39^{c} |
| 200 | 0.39 ± 0.14 | 0.91 ± 0.29 | 1.73 ± 0.11 | 2.46 ± 0.13 | 3.66 ± 0.08^{b} |
| 300 | 0.26 ± 0.18 | 1.34 ± 0.58 | 3.72 ± 0.84 | 5.25 ± 0.75 | 7.62 ± 1.16^{a} |

Notes:
a, b, and c represented significant differences in a cumulative increase of lateral branches per centimeter under different light intensities on the 10^{th} day.

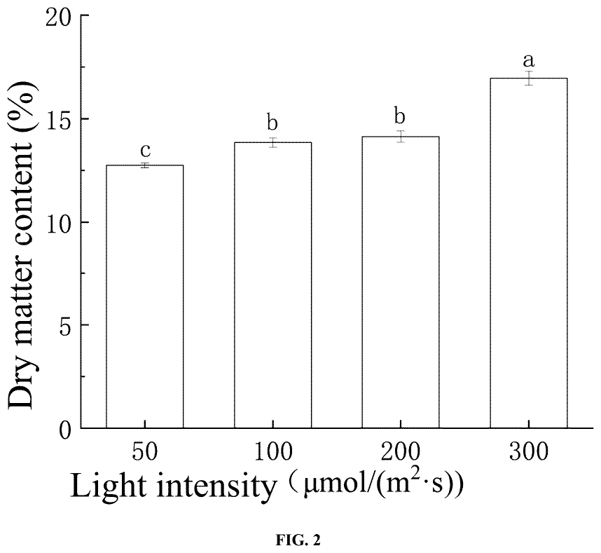

The dry matter content on the 10^{th} day was shown in Table 2 and FIG. 2. With the increase of light intensity, the dry matter content of algae showed an upward trend. The dry matter content of algae at a light-photon flux of 300 μmol·m⁻²·s⁻¹ reached 16.950/60.34%, which was significantly higher than that under other light intensities (P<0.01). This indicated that the high light-photon flux of 300 μmol·m⁻²·s⁻¹ could effectively increase the dry matter content of algae.

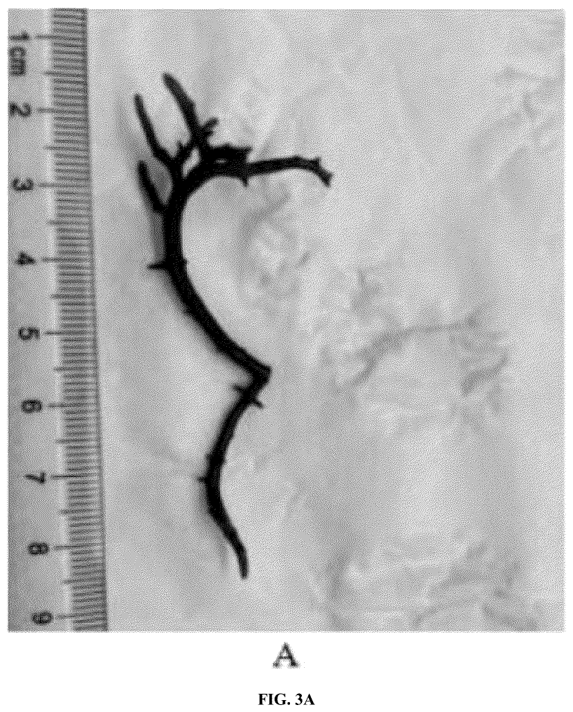

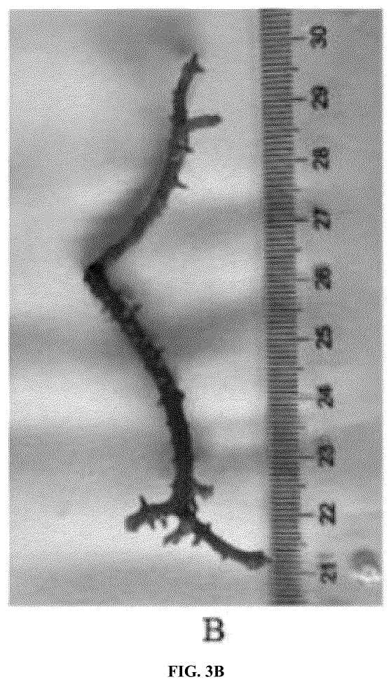

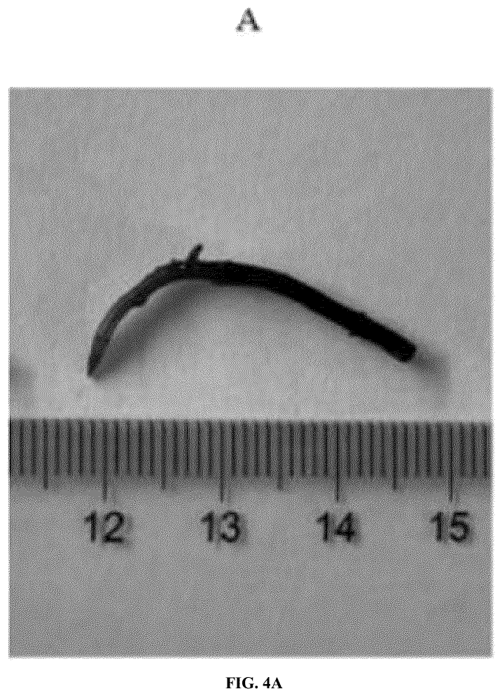

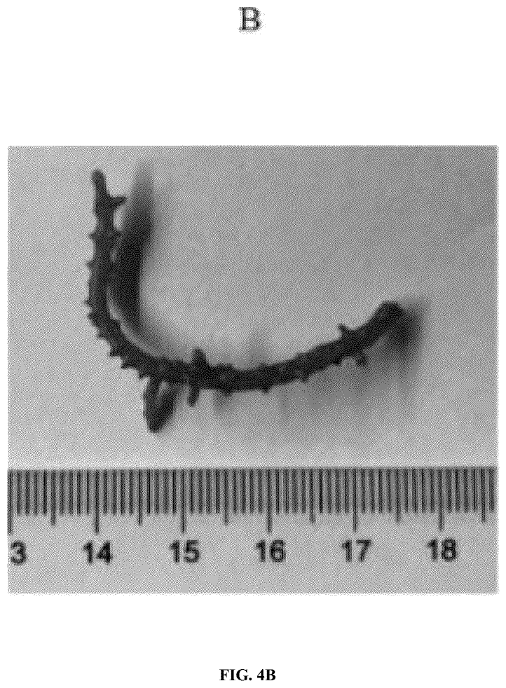

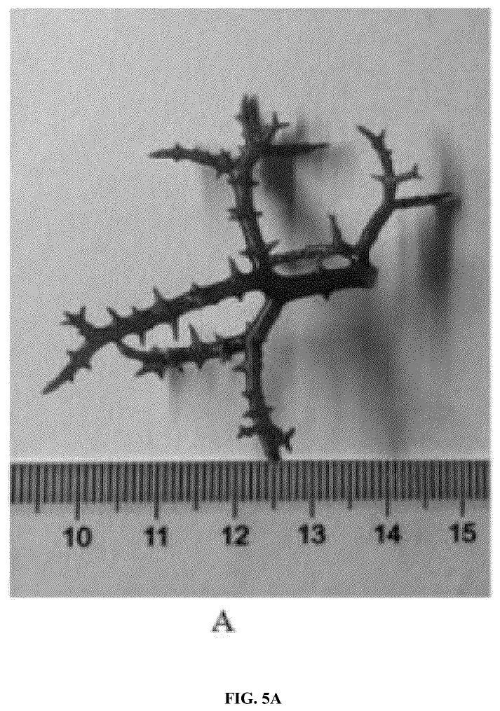

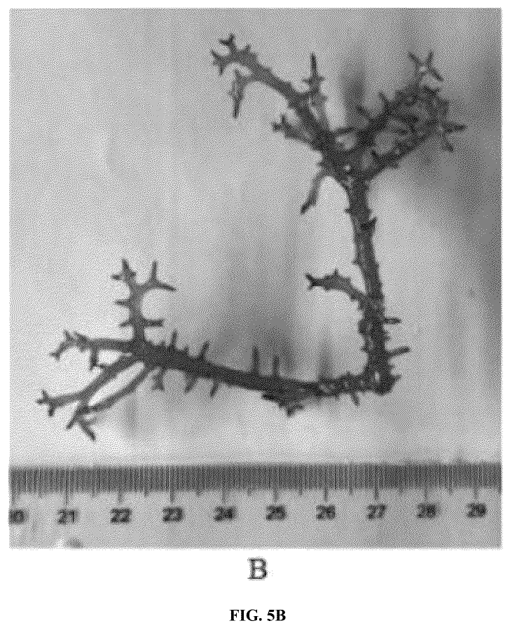

A same K denticulatum plant was cultured for 10 d at a light-photon flux of 50 μmol·m⁻²·s⁻¹ and photographed, and the results were shown in FIGS. 3A-B. This E. denticulatum plant was cultured at a light-photon flux of 300 μmol·m⁻²·s⁻¹ for 10 d, and then photographed, and the results were shown in FIGS. 4A-B. After culturing for 10 d at a light-photon flux of 300 μmol·m⁻²·s⁻¹, the number of lateral branches could be significantly increased. The E. denticulatum was cultured for 15 d at a light-photon flux of 300 μmol·m⁻²·s⁻¹ and photographed. As shown in FIGS. 5A-B, the number of lateral branches was further increased.

TABLE 2
Dry matter content on 10^{th} day under different light intensities

| Light intensity | | | | |
|---|---|---|---|---|
| (μmol · m⁻² · s⁻¹) | 50 | 100 | 200 | 300 |
| Dry matter content (%) | 12.75 ± 0.12 | 13.85 ± 0.23 | 14.13 ± 0.28 | 16.95 ± 0.34 |

The above are merely preferred implementations of the present disclosure. It should be noted that several improvements and modifications may further be made by a person of ordinary skill in the art without departing from the principle of the present disclosure, and such improvements and modifications should also be deemed as falling within the protection scope of the present disclosure.

1. A method for inducing a lateral branch development of Eucheuma denticulatum, comprising the following steps: conducting aeration culture on E. denticulatum under light illumination of 300 μmol·m⁻²·s⁻¹; wherein
the aeration culture is conducted in a Provasoli's enrichment solution (PES) medium;
the light illumination comprises 12 h continuous light and 12 h continuous darkness every day;
the PES medium comprises the following components: 2.35 g/L of NaNO₃, 0.35 g/L of C₃H₇Na₂O₆P·5H₂O, 162.5 mL/L of an ES Fe solution, 162.5 mL/L of a P-II metal solution, and 3.25 g/L of a HEPES buffer;
the ES Fe solution comprises the following components by concentration: 0.7 g/L of Fe(NH₄)₂(SO₄)₂·6H₂O and 0.6 g/L of Na₂EDTA·2H₂O; and
the P-II metal solution comprises the following components by concentration: 1 g/L of Na₂EDTA·2H₂O, 1.14 g/L of H₃BO₃, 0.049 g/L of FeCl₃·6H₂O, 0.164 g/L of MnSO₄·H₂O, 0.022 g/L of ZnSO₄·7H₂O, and 0.0048 g/L of CoCl₂·6H₂O.

2. (canceled)

3. (canceled)

4. The method according to claim 1, wherein the aeration culture is conducted for at least 10 d.
